(19) United States
(12) Patent Application Publication
(10) Pub. No.: US 2012/0221105 A1
(43) Pub. Date: Aug. 30, 2012

(54) PROSTHETIC DEVICE AND METHOD OF MANUFACTURING THE SAME

(75) Inventors: Gregory H. Altman, Arlington, MA (US); Enrico Mortarino, Hickory, NC (US)

(73) Assignee: ALLERGAN, INC., Irvine, CA (US)

(21) Appl. No.: 13/466,686

(22) Filed: May 8, 2012

Related U.S. Application Data
(62) Division of application No. 12/878,757, filed on Sep. 9, 2010.
(60) Provisional application No. 61/241,756, filed on Sep. 11, 2009.

(51) Int. Cl. A61F 2/12

(52) U.S. Cl. 623/8

(57) ABSTRACT

An implantable device for use in tissue and ligament repair comprising at least one knitted section and at least one single continuous fiber traversing the at least one knitted section, the at least one single continuous fiber forming a plurality of traverses extending through the at least one knitted section. The implantable device may comprise at least one single continuous silk fiber. The implantable device is suitable for use in a variety or reconstructive or support applications such as breast reconstruction, mastopexy, breast augmentation revision, breast augmentation support, standard breast augmentation, chest wall repair, organ support, body contouring, abdominoplasty, facial reconstruction, hernia repair, and pelvic floor repair.

### CROSS-REFERENCES TO RELATED APPLICATIONS

This application is a divisional of U.S. patent application Ser. No. 12/878,757, filed Sep. 9, 2010, which claims priority from U.S. Provisional Patent Application No. 61/241,756, filed on Sep. 11, 2009. The disclosures of which are incorporated herein by reference.

### FIELD OF THE INVENTION

The present invention relates to an implantable device for reconstruction or support, and more particularly, to a fabric implantable device for reconstruction or support of tissue and ligaments.

### BACKGROUND OF THE INVENTION

The fields of bioengineering, biomaterials and tissue engineering are providing new options to gradually restore native tissue and organ function through the research and development of scaffolds, meshes, matrices and constructs (i.e. devices) that initially support a disabled tissue or organ, but eventually allow for the development and remodelling of the body's own biologically and mechanically functional tissue. Hence, surgical meshes and scaffolds have been used historically in a variety of applications to reinforce tissue or ligaments where defects or weakness exist. However, certain medical, cosmetic and surgical applications present unique challenges with regard to tissue and ligament repair.

For example, every year, hundreds of thousands of people sprain, tear, or rupture ligaments and tendons of the knee, elbow, hand, shoulder, wrist and jaw. One such ligament is the anterior cruciate ligament (ACL) of the knee. More than 200,000 people in the U.S. alone, tear or rupture their ACL each year. The ACL serves as a primary stabilizer of anterior tibial translation and as a secondary stabilizer of valgus-varus knee angulation, and is often susceptible to rupture or tear resulting from a flexion-rotation-valgus force associated with sports injuries and traffic accidents. Ruptures or tears often result in: severe limitations in mobility; pain and discomfort; and an inability to participate in sports and exercise. Failures of the ACL are classified in three categories: (1) ligamentous, in which ligament fibers pull apart due to tensile stress; (2) failure at the bone-ligament interface without bone fracture; and (3) failure at the bone-ligament interface with bone fracture at the attachment site of bone and ligament. Ligamentous failure is the most common type of ACL failure.

It is widely known that the ACL has poor healing capabilities. Total surgical replacement and reconstruction are required when the ACL suffers a significant tear or rupture. The most common practice is to reconstruct a torn ACL by substituting the torn ligament with the patient's own tissue, also known as an autograft. The middle third of the patellar tendon or the hamstring tendons are commonly used as autografts. Other options for substitute ligaments include donor tissues from a cadaver, also known as allografts, as well as synthetic grafts.

Conventionally, the techniques for reconstructing the ACL involve drilling tibial and femoral tunnels and pulling the autograft, allograft, or artificial ligament through the tunnels. The substitute ligament is then anchored to bone by a mechanical fixation device. Anchors may include the suspensory fixation, staples, as well as interference screws and cross pins. Often, the graft is folded in half to create a double bundle to more closely mimic the native human ACL. The size of the drilled bone tunnels depends on the size of the graft, which in turn depends on the strength of the graft material.

Although the use of autografts is common, the technique is disadvantageously accompanied by morbidity at the second surgery site from which the autograft is taken. For example, stress fracture of the patellar or weakness in the quadriceps muscle may occur, and a long rehabilitation period may be required. Furthermore, the process of harvesting and preparing autogenous tissue prolongs surgery time and causes additional trauma to the patient.

In-growth in the bone tunnels and about the device improves the device's strength and functionality over time. In addition, if sufficient in-growth does not occur, conventional devices may not be able to maintain proper flexibility, integrity, or tension in the long term.

In order to restore the stability of the knee with a replacement ligament, correct tensioning of the graft must also be established and maintained. A common mode of failure for conventional devices occurs when the devices loosen due to bone erosion and degradation around the implant site. In such cases, sufficient in-growth can fail to occur around the device within the bone tunnels. This then results in a slackening of the ligament and an eventual return to a dysfunctional knee.

Another disadvantage with conventional devices includes the release of debris from a failed ligament resulting in chronic inflammation of the joint. A further disadvantage includes osteolysis of bone, in and around the area of ligament attachment. Moreover, device abrasion may occur at the bone tunnel apertures.

In the case of soft tissue repair, surgical meshes and scaffolds are widely used for breast and chest wall reconstruction, strengthening tissues, providing support for internal organs, and treating surgical or traumatic wounds. They are usually made of inert materials and polymers such as Teflon®, polypropylene, polyglycolic acid, polyester, polyglactin 910, etc., although a titanium mesh has been used in some spinal surgeries. But the use of tissue based materials such as acellular dermal matrix (ADM) from human and animal derived dermis is also becoming more popular.

Surgical mesh devices are typically biocompatible and can be made from bioresorbable and/or non-bioresorbable material. For example, Teflon®, polypropylene and polyester are biocompatible and non-bioresorbable while polyglycolic acid and polyglactin 910 are biocompatible and bioresorbable. ADM is processed by removing the cells and epidermis, if applicable, from the donor tissue and leaving only natural biologic components. The most common tissue based materials are human, porcine or equine derived dermal matrix.

One application for soft tissue reconstruction that uses surgical mesh or ADM is breast reconstruction post mastectomy. The aim of breast reconstructive surgery is to restore a woman's breasts to a near normal appearance and shape following the surgical removal of a breast (mastectomy), a crucial step towards emotional healing in women who have been faced with losing their breast as a result of a medical condition such as breast cancer. According to the American Society of Plastic Surgeons (ASPS), approximately 60,000 surgical procedures occur in the U.S. related to non-cosmetic breast reconstruction. Internationally, that number surpasses 80,000 procedures when major industrialized countries are taken into consideration. A contoured, structural and tailored scaffold device designed to meet the unique needs of the breast reconstruction population where a massive loss of tissue occurs and which would work with the body's own immune process to restore the environment to a more natural state would provide a compassionate solution to a significant unmet need.

The breast reconstruction surgical procedure is commonly performed with two different methods, both using ADM as the preferred matrix. The main advantage of ADM over other available surgical meshes is the higher rate of revascularization, providing support and coverage of the defect while preventing infection and capsular contraction. The first method, a one stage reconstruction uses ADM to fully reconstruct the shape of the breast in conjunction with a breast implant at the time of the surgical procedure. The second method, a two stages reconstruction; the first stage consisting of the placement of (a) tissue expander(s) (at the time of mastectomy or later) with ADM to reconstruct the breast; follow by tissue expander expansion with saline solution to expand the muscle and skin tissue; the second and final stage consisting of the replacement of the tissue expander with an implant. In both procedures the pocket for the tissue expander or the implant is created by releasing the inferior origin of the pectoralis muscle and electrocauterizing a subpectoral pocket. A sheet of ADM is centered over the defect and it is sutured to the inframammary fold with continuous or interrupted sutures. The tissue expander or implant is inserted and positioned inside the subpectoralis pocket created. The rest of the ADM is cut to the necessary shape and it is sutured to the inferior edge of the pectoralis muscles, while on the lateral border is sutured to the pectoralis and serratus muscles.

A breast reconstruction procedure alternative to the one described above using ADM is performed with autologous tissue such as TRAM flap. In this surgical procedure, the breast is reconstructed by using a portion of the abdomen tissue group that has been surgically removed, including the skin, the adipose tissue, minor muscles and connective tissue. This abdomen tissue group is taken from the patient's abdomen and transplanted onto the breast site using a similar method as described above with ADM.

The quality of the resulting reconstruction is impacted by subsequent treatment, e.g. post-mastectomy radiation weakens skin tissue, the amount of tissue available e.g. thinner women often lack sufficient tissue, and the overall health and habits, such as smoking, of the individual. Tissue expanders, balloon type devices, are frequently used in an attempt to stretch the harvested skin to accommodate the breast implant. However, harvested tissue has limitations in its ability to conform to the natural breast contour resulting in unacceptable results, including a less than ideal positioning or feel of the breast implant. A scaffold device that can be used as an internal scaffold to act as a “bra” to immediately support a geometrically complex implantation site at the time of surgery would ideally provide the body both the time and structure necessary for optimal healing.

Breast reconstruction in two stages with a tissue expander and ADM followed by the replacement of the tissue expander with an implant has become the most common technique adopted by surgeons. A main advantage is the lengthening of the pectoralis major muscle therefore preventing the commonly referred to as “window shading” after the muscle is released. Another main advantage is the control of the inframammary fold position and shape as well as the lateral breast border.

The use of ADM has advantages against the common surgical mesh devices by lowering the rate of capsular contraction and infection; however despite its low overall complication rate, the procedure is not without risk since ADM can generate a host inflammatory reaction and sometimes present infection. Also, it is very important to note that the properties of ADM are limited to the properties of the tissue that is harvested which can result in variability.

Thus, there is a need for a device or structure that can be used for reconstruction and support that overcomes the disadvantages of known methods and materials.

Furthermore, most biomaterials available today do not possess the mechanical integrity of high load demand applications (e.g., bone, ligaments, tendons, muscle) or the appropriate biological functionality; most biomaterials either degrade too rapidly (e.g., collagen, PLA, PGA, or related copolymers) or are non-degradable (e.g., polyesters, metal), where in either case, functional autologous tissue fails to develop and the patient suffers disability. In certain instances a biomaterial may misdirect tissue differentiation and development (e.g., spontaneous bone formation, tumors) because it lacks biocompatibility with surrounding cells and tissue. As well, a biomaterial that fails to degrade typically is associated with chronic inflammation, where such a response is actually detrimental to (i.e., weakens) surrounding tissue.

If properly designed, silk may offer new clinical options for the design of a new class of medical devices, scaffolds and matrices. Silk has been shown to have the highest strength of any natural fiber, and rivals the mechanical properties of synthetic high performance fibers. Silks are also stable at high physiological temperatures and in a wide range of pH, and are insoluble in most aqueous and organic solvents. Silk is a protein, rather than a synthetic polymer, and degradation products (e.g., peptides, amino acids) are biocompatible. Silk is non-mammalian derived and carries far less bioburden than other comparable natural biomaterials (e.g., bovine or porcine derived collagen).

Silk, as the term is generally known in the art, means a filamentous fiber product secreted by an organism such as a silkworm or spider. Silks produced from insects, namely (i) Bombyx mori silkworms, and (ii) the glands of spiders, typically Nephila clavipes, are the most often studied forms of the material; however, hundreds to thousands of natural variants of silk exist in nature. Fibroin is produced and secreted by a silkworm's two silk glands. As fibroin leaves the glands, it is coated with sericin, a glue-like substance. However, spider silk is valued (and differentiated from silkworm silk) as it is produced as a single filament lacking any immunogenic contaminates, such as sericin.

Unfortunately, spider silk can not be mass produced due to the inability to domesticate spiders; however, spider silk, as well as other silks can be cloned and recombinantly produced, but with extremely varying results. Often, these processes introduce bioburdens, are costly, cannot yield material in significant quantities, result in highly variable material properties, and are neither tightly controlled nor reproducible.

As a result, only silkworm silk has been used in biomedical applications for over 1,000 years. The Bombyx mori specie of silkworm produces a silk fiber (known as a “bave”) and uses the fiber to build its cocoon. The bave, as produced, includes two fibroin filaments or “broins”, which are surrounded with a coating of gum, known as sericin—the silk fibroin filament possesses significant mechanical integrity. When silk fibers are harvested for producing yarns or textiles, including sutures, a plurality of fibers can be aligned together, and the sericin is partially dissolved and then resolidified to create a larger silk fiber structure having more than two broins mutually embedded in a sericin coating.

As used herein, “fibroin” includes silkworm fibroin (i.e. from Bombyx mori) and fibroin-like fibers obtained from spiders (i.e. from Nephila clavipes). Alternatively, silk protein suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

Silkworm silk fibers, traditionally available on the commercial market for textile and suture applications are often “degummed” and consist of multiple broins plied together to form a larger single multi-filament fiber. Degumming here refers to the loosening of the sericin coat surrounding the two broins through washing or extraction in hot soapy water. Such loosening allows for the plying of broins to create larger multifilament single fibers. However, complete extraction is often neither attained nor desired. Degummed silk often contains or is recoated with sericin and/or sericin impurities are introduced during plying in order to congeal the multifilament single fiber. The sericin coat protects the frail fibroin filaments (only ˜5 microns in diameter) from fraying during traditional textile applications where high-through-put processing is required. Therefore, degummed silk, unless explicitly stated as sericin-free, typically contain 10-26% (by weight) sericin.

Sericin is antigenic and elicits a strong immune, allergic or hyper-T-cell type (versus the normal mild “foreign body” response) response. Sericin may be removed (washed/extracted) from silk fibroin; however, removal of sericin from silk changes the ultrastructure of the fibroin fibers.

When typically referring to “silk” in the literature, it is inferred that the remarks are focused to the naturally-occurring and only available “silk” (i.e., sericin-coated fibroin fibers) which have been used for centuries in textiles and medicine. Medical grade silkworm silk is traditionally used in only two forms: (i) as virgin silk suture, where the sericin has not been removed, and (ii) the traditional more popular silk suture, or commonly referred to as black braided silk suture, where the sericin has been completely removed, but replaced with a wax or silicone coating to provide a barrier between the silk fibroin and the body tissue and cells. Presently, the only medical application for which silk is still used is in suture ligation, particularly because silk is still valued for it mechanical properties in surgery (e.g., knot strength and handlability).

Therefore, there also exists a need to generate a silk-based implantable device that is biocompatible and promotes ingrowth of cells.

### SUMMARY OF THE INVENTION

In view of the foregoing, embodiments according to aspects of the present invention address the disadvantages of conventional devices and provide a support structure or an improved device for reconstruction of tissue.

The implantable device of the present invention is suitable for use in a variety or reconstructive or support applications including, but not limited to, breast reconstruction, mastopexy, breast augmentation revision, breast augmentation support, standard breast augmentation, chest wall repair, organ support, body contouring, abdominoplasty, facial reconstruction, hernia repair, and pelvic floor repair.

Advantageously, certain embodiments provide a reconstructive or prosthetic device having multiple bundles of fibers that closely mimic the natural structure of ligament tissue, such as a native ACL, and allow for new tissue in-growth. In addition, embodiments provide sufficient flexibility to enable implementation in a large range of anatomies.

Furthermore, embodiments may be compatible with conventional anchoring systems and may be implanted into the same footprint as the native ligament tissue. Additionally, embodiments enable equal tensioning and distribute load evenly across a reconstructive device while it sustains a physiologic load. Another aspect of embodiments is the minimization of abrasion at bone tunnel apertures or elsewhere. Embodiments also endure surgical procedures without sustaining damage, such as unraveling or changes in critical dimensions, when being pulled through bone tunnels.

An example embodiment provides a knitted prosthetic device with at least one knitted section and at least one single continuous fiber traversing the at least one knitted section. The at least one single continuous fiber forms a plurality of traverses extending through the at least one knitted section. It is configured to support a load applied across the prosthetic device.

In the example embodiment, the plurality of traverses may be organized into a plurality of spaced bundles.

In the example embodiment, the at least one knitted section may include two knitted sections, where the at least one single continuous fiber extends between the two knitted sections and the two knitted sections are longitudinally separated by an intermediate section defined by the plurality of traverses. Furthermore, the intermediate section may be less dense than the two knitted sections, and as such, the intermediate section may provide a shapeable end for the prosthetic device when the prosthetic device is folded transversely across the intermediate section. The shapeable end facilitates the positioning of the prosthetic device for implantation, particularly when the prosthetic device must be guided through a channel in bone or tissue. In some embodiments, the intermediate section is tapered, e.g., like a bullet head, when the prosthetic device is folded transversely across the intermediate section.

In the example embodiment, the at least one single continuous fiber may extend beyond the at least one knitted section and form loops at opposing ends of the prosthetic device.

In the example embodiment, the ends of the prosthetic device may be anchored to allow the at least one knitted section to act as a ligament. For example, embodiments may include features, such as loops and/or button holes, that accommodate device tensioning and/or a variety of fixation or anchoring devices that act to keep the prosthetic device in position. Once the prosthetic device is anchored, the at least one single continuous fiber provides the required load bearing support.

Additionally, embodiments may be constructed from a strong polymer, preferably, but not limited to, silk, where the polymer is bioresorbable to allow substantial in-growth, both in the device itself and within and around the bone tunnels to maintain or improve the strength of the device-tissue construct over time. Embodiments employing such a polymer exist long enough in the joint to support the knee prior to tissue in-growth, but bioresorb as load bearing responsibilities are transferred over to the newly developing tissue.

Although some embodiments described herein may be specifically applied as prosthetic ligaments for reconstructing an ACL, it is understood that embodiments according to aspects of the present invention may be employed for other tendons and support structures. Still other aspects, features, and advantages of the present invention are readily apparent from the following detailed description, by illustrating a number of exemplary embodiments and implementations, including the best mode contemplated for carrying out the present invention. The present invention is also capable of other and different embodiments, and its several details can be modified in various respects, all without departing from the spirit and scope of the present invention. Accordingly, the drawings and descriptions are to be regarded as illustrative in nature, and not as restrictive.

### BRIEF DESCRIPTION OF THE FIGURES

FIG. 1 illustrates an example prosthesis employing five sections (knit sections, a weft exposed section, loops) according to aspects of the present invention.

FIG. 2 illustrates a folded configuration for the example prosthesis of FIG. 1.

FIG. 3 illustrates a front view of the example prosthesis of FIG. 1 when implanted as a canine ACL prosthesis.

FIG. 4 illustrates a rear view of the example prosthesis of FIG. 1 when implanted as a canine ACL prosthesis.

FIG. 5 illustrates an intra-articular view of the example prosthesis of FIG. 1 when implanted as a canine ACL prosthesis.

FIG. 6 illustrates an example prosthesis employing a knitted sock and a cuff section according to aspects of the present invention.

FIGS. 7A-D illustrate an example technique for configuring a prosthesis according to aspects of the present invention.

FIG. 8 illustrates an internal sectional view of a human breast with the device of the present invention implanted.

### DETAILED DESCRIPTION OF THE PREFERRED EMBODIMENTS

Aspects of the present invention relate to the repair of specific bodily tissues, such as hernia repair, urinary bladder tissues and slings, pelvic floor reconstruction, peritoneal wall tissues, vessels (e.g., arteries), muscle tissue (abdominal smooth muscle, cardiac), hemostats, and ligaments and tendons of the knee and/or shoulder as well as other frequently damaged structures due to trauma or chronic wear. Examples of ligaments or tendons that can be produced include anterior cruciate ligaments, posterior cruciate ligaments, rotator cuff tendons, medial collateral ligaments of the elbow and knee, flexor tendons of the hand, lateral ligaments of the ankle and tendons and ligaments of the jaw or temporomandibular joint. Other tissues that may be produced by methods of this disclosure include cartilage (both articular and meniscal), bone, skin, blood vessels, stents for vessel support and/or repair, and general soft connective tissue.

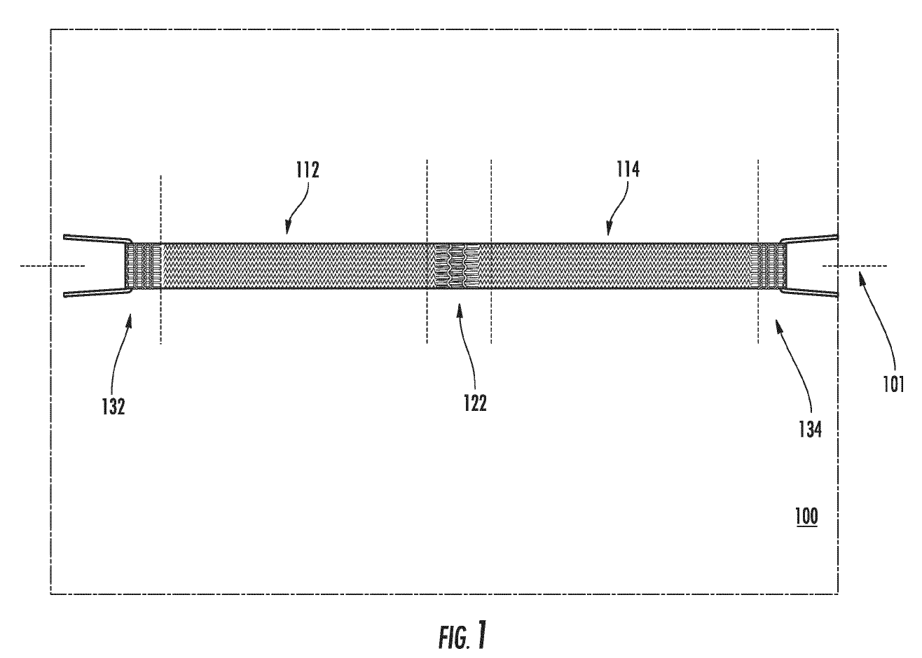

Referring to the figures, FIG. 1 illustrates a first embodiment of a ligament prosthesis 100 having five sections 132, 112, 122, 114, and 134 arranged along a longitudinal axis 101. The ligament prosthesis 100 has two knitted sections 112 and 114, which are separated by a weft exposed section 122. Meanwhile, loops 132 and 134 are disposed on the ends of the prosthesis 100. In some aspects, the knitted sections 112 and 114 act as the ligament portion of the prosthesis 100, although it is understood that load bearing structures extend through the entire prosthesis 100 as described further below. In addition, as described further below, the weft exposed section 122 and the loops 132 and 134 are provided for positioning and anchoring the prosthesis 100 during implantation.

In some preferred embodiments, the ligament prosthesis 100 is generally formed of polymers, such as protein biomaterials. In particular, embodiments may be formed from silk, such as Bombyx mori silkworm silk fibroin. The excellent strength of the material permits less material to be employed in the ligament prosthesis 100, while still meeting the mechanical requirements of functional ACL tissue, for example.

The use of silk in similar prosthetic devices is described further in U.S. patent application Ser. No. 12/052,719, filed Mar. 20, 2008 and entitled “PROSTHETIC DEVICE AND METHOD OF MANUFACTURING THE SAME,” the contents of which are incorporated entirely herein by reference. The silk fibers suitable for use in the present invention are biocompatible due primarily to the extraction of sericin from the silkworm silk fibers and are substantially free of sericin. As a result, the sericin-extracted silk fibroin fibers are non-immunogenic; i.e., they do not elicit a substantial allergic, antigenic, or hyper T-cell response from the host, diminishing the injurious effect on the surrounding biological tissues, such as those that can accompany immune-system responses in other contexts. In addition, the ingrowth of cells around the sericin-extracted silk fibroin fibers is promoted and the sericin-extracted silk fibroin fibers are substantially biodegradable. Indications that the device or structure is “substantially free” of sericin means that sericin comprises less than 20% sericin by weight. Preferably, sericin comprises less than 10% sericin by weight. Most preferably, sericin comprises less than 1% sericin by weight. Furthermore, “substantially free” of sericin can be functionally defined as a sericin content that does not elicit a substantial allergic, antigenic, or hyper T-cell response from the host. Preferably, the silk fibers suitable for use in the present invention are sericin-extracted silk fibroin fibers that substantially retain their native protein structure and have not been dissolved and reconstituted.

It should be appreciated that the prosthetic devices of the present invention, are not limited to the use of silk, and may be formed from a strong polymer that is capable of being knitted. In particular, the polymer is preferably bioresorbable to allow substantial in-growth, both in the device itself and within and around the bone tunnels to maintain and to improve the strength of the device-tissue construct over time. Moreover, embodiments may be processed with a surface treatment, which increases material hydrophilicity, biocompatibility, and handling for ease of cutting and graft pull-through, as well as anti-microbial and anti-fungal coatings.

In some embodiments, the use of silk may be combined with another material to enhance the material properties of the yarn. For example, embodiments may be formed with yarn made from a combination of silk yarn and a man-made yarn such as UHMWPE (Ultra High Molecular Weight Polyethylene, also known under the commercial name Dyneema®). The use of UHMWPE, for example, enhances the mechanical properties of the embodiments per cross sectional area.

In general, embodiments of the present invention employ materials which that have undergone extensive biocompatibility testing in accordance with the ISO-10993 recommendations for a permanent implantable device. Generally, the materials are biocompatible, non-cytotoxic, non-irritating, non-toxic, non-pyrogenic, non-mutagenic, non-clastogenic, non-hemolytic, and non-antigenic with no evidence of sensitization or complement activation.

Referring again to FIG. 1, the knitted sections 112 and 114 may be formed from a weft-knitted fabric or a warp-knitted fabric. In particular, the fabric may employ yarns formed from silk fibers. Weft yarns traverse from the knitted section 112 to the knitted section 114 and provide the load bearing features of the prosthesis 100. A plurality of traverses may extend through and between the knitted sections 112 and 114. The organization and distribution of the weft yarns are determined by the knit structure of the knitted sections 112 and 114. For example, in a particular prosthesis studied for use as a canine ACL, the weft yarns are organized into distinct bundles of four traverses from one knitted section to the other. Each bundle of traverses acts independently to provide the load bearing function. It has been discovered that the organization of weft yarns, e.g., the number of yarns in distinct bundles and the spacing between the bundles, determines the amount of abrasion applied by the prosthesis 100, against surrounding bone, for example, when implanted. Thus, an optimal configuration for the weft yarns can be determined to minimize undesired abrasion.

Each of the knitted sections 112 and 114 has one or more rows, also known as courses. One or more single continuous weft yarns are laid into, or received by, one or more courses in each knitted section 112 and 114 and traverse across the knitted sections 112 and 114. In addition, the one or more single continuous weft yarns extend between and connect the knitted sections 112 and 114 form traverses that define the weft exposed section 122. The organization of the one or more weft yarns in the weft exposed section 122 corresponds generally with their organization in the knitted sections 112 and 114. For example, in the particular prosthesis studied for use as a canine ACL described previously, the weft yarns in the weft exposed section 122 are also organized in distinct bundles of four traverses. The structure of the weft exposed section 122 generally has a lower density than the structure of the knitted sections 112 and 114.

A single continuous weft yarn in the ligament prosthesis 100 refers to a single yarn that traverses the knitted sections 112 and 114 and the weft exposed section 122 to form a plurality of traverses extending between the knitted sections 112 and 114. For instance, the continuous yarn may be laid in and traversed in a repetitive S-shaped or Z-shaped pattern, extending from the first knitted section to the second knitted section where it pivots and returns back to the first knitted section where it may pivot again and repeat the sequence several times. In other words, each traverse of the single continuous yarn is a continuation of the previous traverse extending in the opposite longitudinal direction. In this way, a single continuous yarn with more than one traverse may connect the two knitted sections 112 and 114 and form the weft exposed section 122. The pivot points are not limited to any one location.

Of course, the ligament prosthesis 100 may contain more than one single continuous yarn laid in and traversed in a repetitive pattern extending between the first knitted section and the second knitted section, thereby connecting the two knitted sections 112 and 114 with more than one traverse from more than one single continuous yarn. The traverses of the multiple single continuous yarns may extend in the same direction, opposite directions, or any combination thereof.

The one or more single continuous yarns form a plurality of parallel, non-discrete, and non-independently translatable elements which may be organized into bundles, i.e., a plurality of longitudinal fibers. Advantageously, non-independently translatable fibers enable equal tensioning of all of the parallel elements of the ligament prosthesis 100 when a force acts to draw the knitted sections 112 and 114 apart.

If the ligament prosthesis 100 is a weft knitted fabric, each traverse of a single continuous yarn is laid into a course of at least one knitted section and tucked, or secured, at least one time in the course. The tucking effectively locks the continuous yarn into the knitted section, minimizing sliding of the yarn with respect to the knitted section, or vice versa, and minimizing any changes in the dimensions of the weft exposed section 122. One or more traverses of the single continuous yarn may be laid into and/or tucked into a single course of the knitted section to form bundles of the traverses, before transitioning to the next course, or row, of the knitted section. In general, the knitted sections 112 and 114 may contain one or more rows, where each row has one or more traverses from a single continuous yarn.

Although the embodiments described herein may include a yarn tucked at each knit section, it is noted that knits that are not made on a weft knitting machine do not necessarily require that each yarn be tucked in each knit section. As such, embodiments of the present invention are not limited to those that tuck the continuous yarn at each knit section.

The longitudinal edges of knitted sections 112 and 114, are also finished to prevent unraveling of the knit. In one embodiment, the edges are binded off. Various stitch formations may be used for this purpose. Due to this binding, no further processing, such as twisting of the prosthesis 100, is generally required. Alternatively, unraveling may be prevented by employing densely knitted sections at the edges. In this alternative embodiment, the edges are finished when the densely knitted sections are cut sharply.

The loops 132 and 134 are defined by a knit structure at the ends of sections 112 and 114, respectively, and the weft yarns, which wrap around to form an open loop.

Aspects of the knitted sections 112 and 114 as well as the weft yarns and other aspects of the ligament prosthesis 100 are also taught in U.S. patent application Ser. No. 12/052,719, described previously.

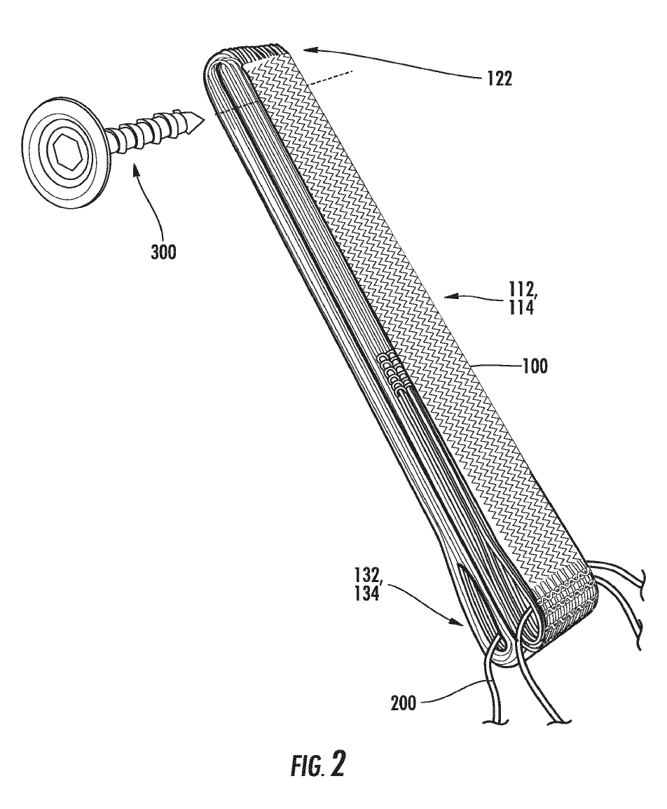

As shown in FIG. 2, the prosthesis 100 may be folded transversely at the weft exposed section 122, so that the two knitted sections 112 and 114 are coincident and the two loops 132 and 134 are coincident. When the prosthesis 100 is in this configuration, the weft exposed section 122 defines one end of the prosthesis 100 and the loops define the other end of the prosthesis 100.

As described previously, the structure of the weft exposed section 122 has a lower density than the structure of the knitted sections 112 and 114. This lower density of the weft exposed section 122 facilitates the folding of the prosthesis 100 as shown in FIG. 2. In addition, the contrasting structure of the weft exposed section 122 provides a convenient visual indication of where the fold should be made. Moreover, the structure of the weft exposed section 122 allows one end of the prosthesis 100 to be shaped in a manner that makes it easier to guide the end of the prosthesis 100 through a bone tunnel or tissue. For example, the weft exposed section 122 may be temporarily shaped like a bullet head. Furthermore, the weft exposed section 122 may be used to facilitate the engagement of the device with a fixation device, such as a screw 300 as shown in FIG. 2. For example, in an over the top fixation procedure, the weft exposed section 122 may conform easier to accommodate a post, screw, suture, or the like. Alternatively, in a through tunnel fixation procedure, the weft exposed section 122 may form a loop to accommodate the EndoButton® CL (Smith & Nephew, MA), interference screw, cross pin, or the like. Various fixation techniques and devices for the prosthesis 100 are further taught in U.S. patent application Ser. No. 12/052,719, described previously.

As further illustrated in FIG. 2, the loops 132, 134 at one end may be used to engage the prosthesis 100 with sutures 200, or with other fixation devices, such as a post or screw. In addition, the loops 132, 134 may be used to engage the prosthesis 100 for pretensioning in preparation of anchoring the prosthesis 100. Furthermore, the loops 132, 134 may be used to engage the prosthesis 100 to pull the prosthesis 100 trough bone tunnel. The loops 132, 134 may be used to anchor the prosthesis 100 to tibial bone, so they may be referred to as tibial loops.

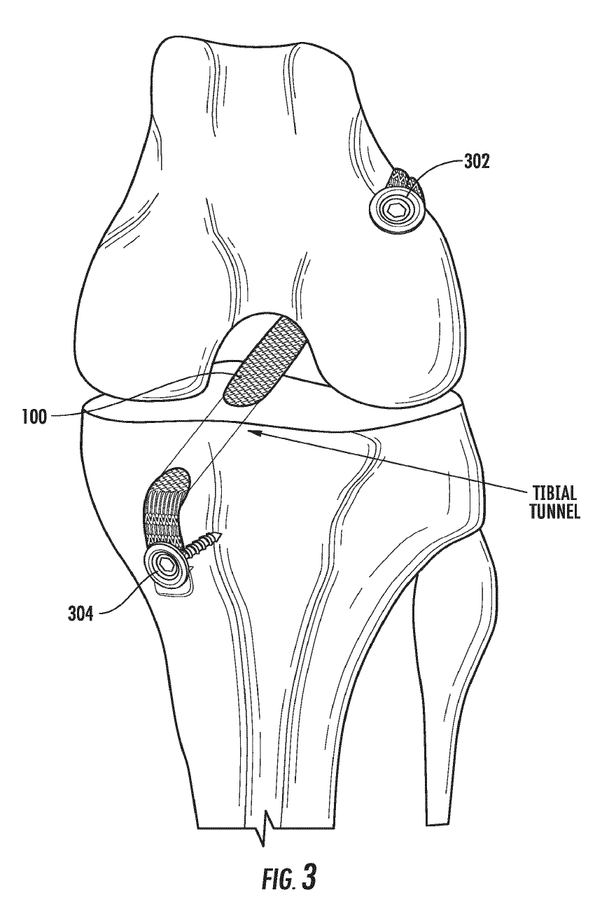

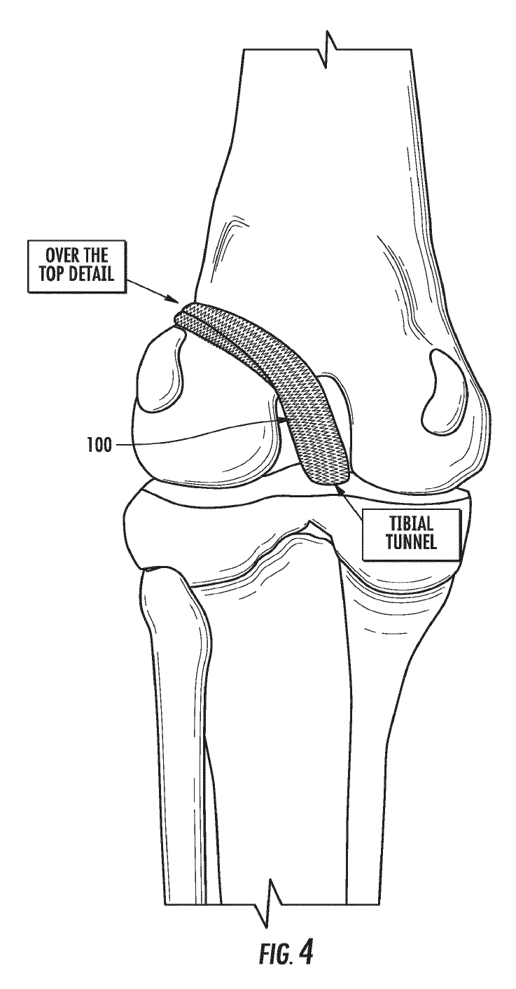

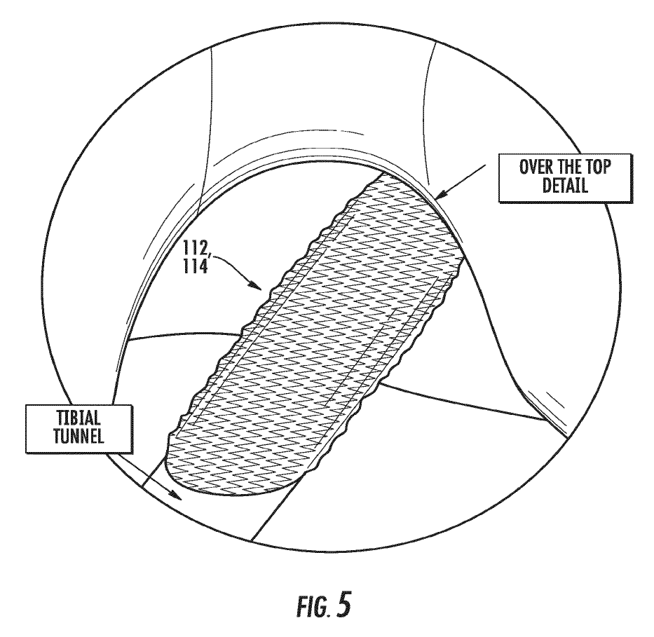

In an example application of the prosthesis 100, FIGS. 3-5 illustrate an embodiment of the prosthesis 100 implanted in a canine knee with an over the top procedure. FIG. 3 illustrates a front view of the prosthesis 100; FIG. 4 illustrates a rear view of the prosthesis 100; and FIG. 5 illustrates an intra-articular view of the prosthesis 100. As FIGS. 3-5 show, the knitted sections 112 and 114 serve as a functional ligament when one end, i.e., the weft exposed section 122, is anchored to the femoral bone by a screw (with washer) 302 and the other end, i.e., the loops 132, 134, are anchored to the tibial bone by a screw (with spiked washer) 304. The knitted section 112 and 114 of the prosthesis 100 allow full functioning of the knee joint while providing the necessary scaffolding and void volume for organized tissue in-growth and remodeling. As described above, the one or more single continuous weft yarns connecting the knitted sections 112 and 114 provide the load bearing function for the prosthesis 100.

Designed to serve as a transitory scaffold that provides immediate knee joint stabilization following surgical repair, embodiments leverage the patient's own internal repair machinery and growth environment to engineer new viable ligament tissue. While conducting the body's own regenerative processes at the implant site, the transitory nature of the graft is designed to allow for tissue healing and rejuvenation as the scaffold becomes less necessary and is bioresorbed through natural and localized tissue remodeling. This approach anticipates and utilizes the body's own inflammatory, revascularization, healing, mechanical and remodeling cascades which follow graft implantation without notice to the patient.

Although the embodiments described herein include the weft exposed section 122 and the loops 132, 134, it is understood that embodiments of the present invention can take advantage of the load bearing features of the weft yarns without these sections. For example, an embodiment may include a single knitted section, rather than two knitted sections separated by a weft exposed section. This single knitted section includes weft yarns, but is positioned and anchored without the benefit of a weft exposed section and loops.

Other variations consistent with aspects of the present invention:

1. A prosthesis that employs one section having a homogeneous construction from one end to the other. This prosthesis may be used flat or folded in half. The homogeneous construction may be formed by a continuous loop or by a knit section.

2. A prosthesis that employs two sections and having two distinct constructions. This prosthesis may be used flat or folded in half. This prosthesis may be formed by a combination of at least two of the following structures: a loop, a knitted section, or an exposed weft section.

3. A prosthesis employing three sections and having two or more distinct constructions. This prosthesis may be used flat or folded in half. This prosthesis may be formed by a combination of at least two of the following structures: a loop, a knitted section, or an exposed weft section.

4. A prosthesis employing four sections and having two or more distinct constructions. This prosthesis may be used flat or folded in half. This prosthesis may be formed by a combination of at least two of the following structures: a loop, a knitted section, or an exposed weft section.

5. A prosthesis employing five sections and having two or more distinct constructions. The device may be used flat or folded in half along the longitudinal length. This prosthesis may be formed by a combination of at least two of the following structures: a loop, a knitted section, or an exposed weft section.

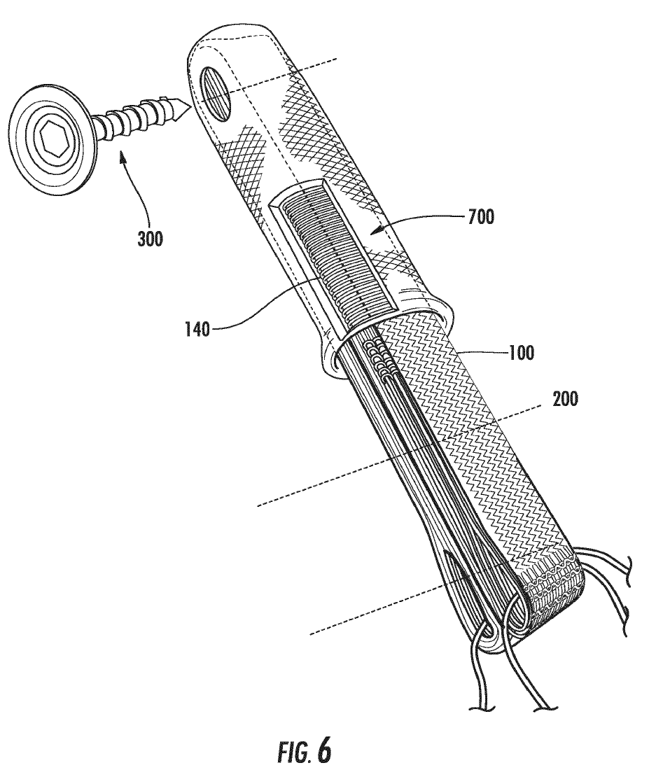

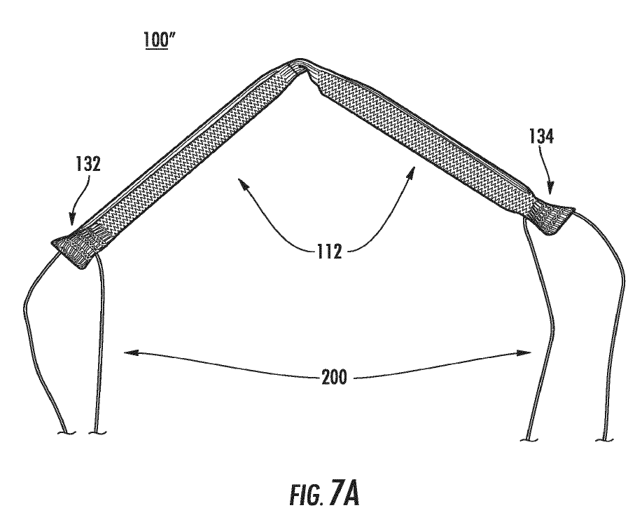

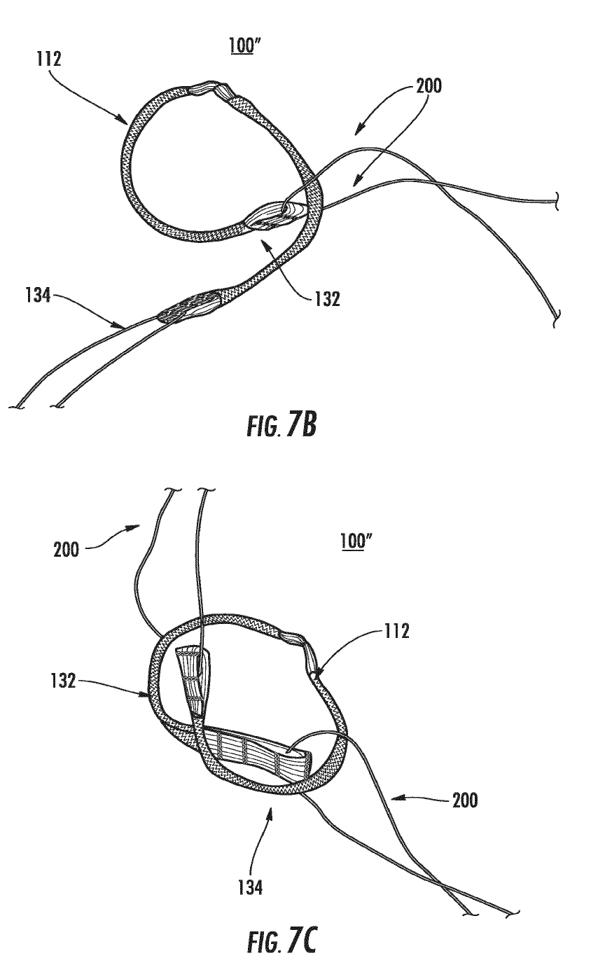

Further embodiments according to aspects of the present invention may employ additional features to minimize abrasion from contact with bone surfaces. For example, as shown in FIG. 6, a knitted sock 700 may be fitted over a ligament prosthesis 100′ similar to the ligament prosthesis 100 of FIG. 2. The knitted sock 700 extends along a length of the ligament prosthesis 100. FIG. 6 also shows that the ligament prosthesis 100′ includes a cuff section 140 disposed within the knitted sock 700. The cuff section 140 is a tubular knitted structure that surrounds the yarns of the ligament prosthesis 100′ and provides a knitted barrier to protect the yarns from abrasion from the bone surfaces. The cuff section 140 may be employed at any section of the ligament prosthesis 100′ to minimize possible abrasion. As shown in FIG. 6, the knitted sock 700 may be used in combination with the cuff section 140 to provide enhanced protection from abrasion. Aspects of the knitted sock and the cuff section are further taught in U.S. patent application Ser. No. 12/052,719, described previously.

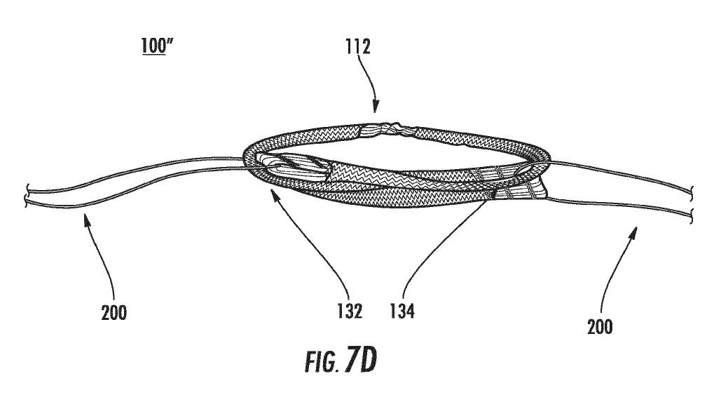

In further embodiments, FIGS. 7A-D illustrate an example technique for folding a ligament prosthesis 100″ according to aspects of the present invention. This technique configures the ligament prosthesis into a closed loop, even if the prosthesis is formed from a flat fabric.

FIG. 7A shows a ligament prosthesis 100″ including three sections: two loop sections 132 and 134, one at each end, and a knit section 112 extending from one loop section to the other.

FIG. 7B shows that the ligament prosthesis 100″ is folded a first time to position the end of the loop section 132 approximately one quarter of the prosthesis overall length from the end of the opposite loop section 134. The sutures 200 are passed above and below the knit section 112.

FIG. 7C shows that the ligament prosthesis 100″ is folded a second time to position the end of loop section 134 approximately one quarter of the graft overall length from the end of the opposite loop section 132. The sutures 200 are passed above and below the knit section 112.

FIG. 7D shows that the ligament prosthesis 100″ resembles a closed loop. This configuration is compatible with cross pin type fixation that may be used through the loop section.

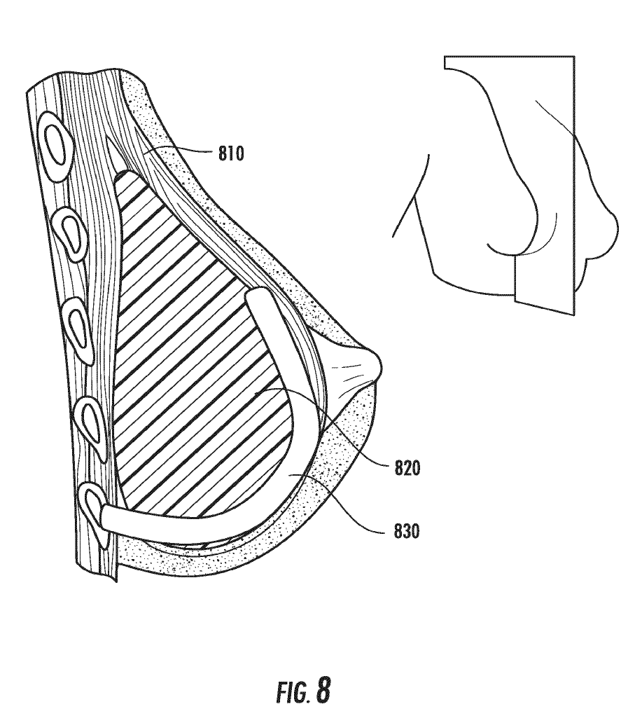

Thus, another aspect of the present invention relates to an implantable device for breast augmentation, breast reconstruction or support. FIG. 8 illustrates an internal sectional view of a human breast with the device of the present invention implanted, for example, to support a prosthetic breast implant. FIG. 8 illustrates the muscle pectoralis major 810, the prosthetic breast implant 820, and the implantable device of the present invention 830.

In the context of breast reconstruction or breast augmentation, the implantable device of the present invention comprises at least one knitted section, and at least one single continuous fiber traversing the at least one knitted section, the at least one single continuous fiber forming a plurality of traverses extending through the at least one knitted section, wherein the implantable device is adapted to engage and support natural breast tissue or a prosthetic breast implant in a patient. In one embodiment, the implantable device includes a portion adapted to be fastened to tissue surrounding the chest cavity of the patient. In one embodiment, the implantable device includes a portion adapted to be fastened to soft tissue surrounding the breast tissue or the prosthetic breast implant. In one embodiment, the implantable device includes a portion adapted to be fastened to a boney structure adjacent to the breast tissue or the prosthetic breast implant. In one embodiment, the implantable device is formed in a predefined shape adapted to conform to at least a portion of a region of natural breast tissue or a breast implant. In one embodiment, the implantable device includes factors for promoting in-growth of breast tissue. In one embodiment, the implantable device, when implanted, at least partially replaces breast connective tissue. In one embodiment, the implantable device is formed in a sling shape to provide support for a breast or a breast implant when the implantable device is implanted in a patient. In one embodiment, the implantable device is formed in an elongated shape to provide support in an inframammary region of a breast when the implantable device is implanted in a patient. In one embodiment, the implantable device is formed in a cup shape to provide inferior support in an inframammary region of a breast when the implantable device is implanted in a patient. In one embodiment, the implantable device is formed in a cup shape to provide medial or lateral support for the breast when the implantable device is implanted in a patient. In another embodiment, the implantable device may incorporate at the end of loop sections 132 and 134, for example, needles to provide minimally invasive insertion and positioning in breast support application.

Still yet another aspect of the present invention relates to a method of supporting breast tissue or a breast implant in a patient comprising, providing an implantable device for a breast augmentation or reconstruction procedure, the implantable device comprising: at least one knitted section, and at least one single continuous fiber traversing the at least one knitted section, the at least one single continuous fiber forming a plurality of traverses extending through the at least one knitted section, and inserting the implantable device between the skin of the patient and the breast tissue or the breast implant. In one embodiment, the method further comprises fastening the implantable device to tissue surrounding the chest cavity of the patient. In one embodiment, the method further comprises fastening the implantable device to soft tissue surrounding the breast tissue or the prosthetic breast implant. In one embodiment, the method further comprises fastening the implantable device to a boney structure adjacent to the breast tissue or the prosthetic breast implant. In one embodiment, the method further comprises forming the implantable device into a predefined shape adapted to conform to at least a portion of a region of natural breast tissue or a breast implant. In one embodiment, the method further comprises treating the implantable device with factors for promoting in-growth of breast tissue. In one embodiment, the implantable device is inserted in an inframammary region of the breast to provide vertical positioning of the breast and reduce vertical inferior displacement of the breast. In one embodiment, the implantable device is inserted in a medial side of the breast to provide medial positioning of the breast and reduce medial displacement of the breast. In one embodiment, the implantable device is inserted in a lateral side of the breast to provide lateral positioning of the breast and reduce lateral displacement of the breast.

The methods described herein can be used to generate an implantable device that possesses sufficient strength to resist loads experienced upon implantation into the patient and thereby provide support to the adjacent tissue. The implantable device used can be designed to possess one or more biomechanical properties of the breast tissue (e.g., soft tissue such as connective tissue) to allow for adequate load resistance. Furthermore, the implantable device supports the in-growth of cells. The more closely the implantable device mimics the biomechanics of the native tissue, the more closely the generated tissue will resemble native tissue.

The implantable device of the present invention can be produced in a variety of sizes and shapes. In one embodiment, the implantable device serves as a scaffold for tissue generation within the breast at the site of implantation. The new tissue generated can serve as an integral component of the breast repair/augmentation, and/or an aid in recovery from the incisions made during the surgery (e.g., breast reconstruction, breast augmentation, mastopexy). The specific size, shape, and fiber organization of the implantable device will vary with respect to the type of procedure and the specific use of the fabric in that procedure, and can be determined by the skilled practitioner for each individual patient. In one embodiment, the device is designed so that it can at least partially replace breast connective tissue in the patient (e.g., tissue that was lost due to surgical removal or otherwise damaged).

The implantable device can take the form of one or more components designed to resemble and replicate native tissue components within the breast, described herein. The implantable device can be designed to replicate one specific tissue structure, or can resemble a plurality of tissue structures (e.g., that are normally found closely associated or interconnected within the breast).

In one embodiment the implantable device is designed to replace or replicate connective tissue that spans the breast area and connects the fascia and/or skin (e.g., connective retinaculum, fascia mammae, fibrous lamella). The implantable device can be designed to have one or more biomechanical properties of the fascia of the breast (e.g., superficial fascia, muscular fascia).

The implantable device may additionally comprise one or more components which resemble native tissue components within the breast (e.g., ligament or ligament-like structures).

In one embodiment, the implantable device fabric is designed and implanted to support the breast structure and/or a prosthesis placed within the breast. The implantable device is adapted to engage the resulting breast (comprising natural breast tissue and/or a prosthetic breast implant). By appropriate placement of the implantable device within the patient, the resulting breast structure is shaped by the implantable device.

The implantable device can be designed to have a variety of different overall shapes (e.g., to conform with the breast tissue when implanted). The implantable device can have a predefined shape that is adapted to conform to at least a portion of a region of natural breast tissue or of a breast implant, within the patient. In one embodiment, the implantable device is sufficiently large to completely or partially cover the lower and/or lateral sections of the breast prosthesis or breast tissue. Such a shape may, for example, allow the implantable device to support the lower pole of the breast prosthesis and/or native breast tissue, emulating the inferior and lateral mammary folds. However, the placement of the implantable device is not limited to any specific location or alignment within the breast, and will depend upon the specific procedure and ultimate goals of tissue construction. In one embodiment, the implantable device is formed in a sling shape (e.g., to provide support for a breast or breast implant, within the patient. In one embodiment, the implantable device is formed in an elongated shape (e.g., to provide support in an inframammary region of the breast, within the patient). In one embodiment, the implantable device is formed in a cup shape (e.g., to provide medial or lateral support for the breast within the patient).

The implantable device may additionally comprise a portion that is adapted to be fastened to the tissue of the patient. This will facilitate implantation. The structure of the portion so adapted will depend upon the means of attachment and/or the place of attachment to the patient. In one embodiment, the attachment is to tissue surrounding the chest cavity of the patient. In one embodiment, the attachment is soft tissue surrounding the breast tissue or surrounding the prosthetic breast implant. In one embodiment, the attachment is to a boney structure adjacent to the breast tissue, or adjacent the prosthetic breast implant.

The implantable device may additionally include one or more agents that promote in-growth of cells to thereby generate new breast tissue. Such agents include, without limitation, cell attachment factors, growth factors, attachment promoting materials, drugs, and chemoattractants.

Breast Anatomy

The inframammary fold is the natural boundary of the breast from below where the breast and the chest meet. The inframammary fold is located at the fifth-sixth rib. The lowest portion extends to the sixth intercostal space. This fold has a constant position.

The inframammary region contains a number of thick collagen fibers, stretched between superficial fascia and deep fascia. The superficial fascia is made up of both collagen and elastic fibers. The superficial fascia of the female breast subcutaneous fascial system is exceptionally thick. The superficial fascia connects to the deep fascia (muscular fascia) through thickened retinaculum along the sternum. A connective band known as the anterior breast capsule (fascia mammae) detaches from the superficial fascia. This fascia, and the fascia of the subclavian area support the mammary gland by means of their retinaculum fibrosa (Cooper's ligaments). Cooper's ligaments and also fascia mammae detach from the superficial fascia and connect to the skin (the deep dermis). The inframammary retinaculum originate from the superficial fascia, and consists of merging dense connective retinaculum. The superficial fascia is separated from the muscular fascia through a thin, deep, subcutaneous layer where the connective retinacula are almost horizontal. They are joined by elastic septa which include adipose lobules. In thin women there are only a few of these and they are small, and fixed to the deeper muscular fascia. There is fusion of the superficial fascia with the deeper fascia, along the sternum. Medially the superficial fascia merges into the anterior membrane of the sternum and is composed of fibers coming from the tendinous apparatus of the sternocleidomastoid and pectoralis major muscles.

A transverse fibrous lamella comes off the fascia almost at the 6^{th} rib, and extends the full length of the inframammary crease. This structure has a different texture and a denser consistency from the superficial fascia. Between the superficial and deep fascia, there is a layer consisting of fibroareolar tissue and occasionally fibrofatty tissue. At the submammary area, the tissue is more fibrous at the sixth rib-sixth intercostal space. The superficial fascia connects with the deeper muscular fascia by means of thicker retinaculum at the deep inframammary subcutaneous layer. The superficial fascia here is adherent to the deep plane (muscular fascia) and more resistant to fraction. The adherence is histologically made up of multiple, short, fibrous connections which do not pass through the fibromuscular plane.

Mammary ligaments form a circumferential ligament about the breast to form a circumferential fusion between the superficial fascia and the deep fascia. This connective ligament which completely surrounds the breast to form a circular boundary to the cleft between the superficial fascia and deep fascia is often referred to as the circumferential mammary ligament. The circumferential mammary ligament forms a natural boundary connecting two tissue layers that a surgeon dissecting between the layers may use to define and limit the extent of the dissection. These defined layers also offer a region for tissue growth, as disclosed in U.S. Patent Application Publication 2008/0300681.

### Use in Breast Surgery

Implantable devices designed to serve as tissue supports and/or scaffolds for breast reconstruction may be used in a wide range of procedures involving breast augmentation or mastopexy, including, for example, in breast lift procedures, breast augmentation procedures, in post-mastectomy reconstruction.

One aspect of the invention relates to the use of the implantable device described herein in a method for supporting a breast structure within a patient. The method involves positioning the implantable device within the patient in a supporting position relative to the breast structure. The breast structure may comprise native breast tissue (e.g., a mammary gland), or a breast prosthesis (e.g., a breast implant), or a combination thereof. Generally this involves implanting the implantable device at an anatomical location between the skin covering the breast tissue and the breast tissue and/or breast implant to be supported within the patient. The specific position (e.g., depth) between the skin and the supported tissue will vary with the actual procedure, and can be determined by the skilled practitioner. In one embodiment, positioning the implantable device comprises covering the lower and lateral sections of the breast area. In one embodiment, the implantable device is inserted in a medial side of the breast to support medial positioning of the breast and/or implant, and reduce medial displacement of the breast and/or implant. In one embodiment, the implantable device is inserted in a lateral side of the breast to support lateral positioning of the breast and/or implant, and reduce lateral displacement of the breast and/or implant. In one embodiment, the implantable device comprises one or more biomechanical properties of a tissue that would be present naturally in the breast at such a supportive position.

Methods for using supportive matrices as surgical tools are well known in the art and can be applied to the implantable device described herein by the skilled practitioner. Implanting the implantable device typically involves inserting the implantable device and fixing the matrix in the desired position. Such methods typically involve fixation of the matrix in the desired position (e.g., across the lower and lateral sections of the breast to support the lower pole of a breast prosthesis/breast tissue, or on the lateral or medial side of the breast to inhibit lateral or medial displacement). Fixation or attachment may be achieved using any suitable method known in the art, for example, by placement of sutures or staples, or with use of a tacking device. Appropriate methods for attachment of the implantable device described herein, during the implantation procedure, is to be determined by the skilled practitioner. The implantable device described herein can be attached to bone (e.g., one or more ribs), muscle, or soft tissue. In one embodiment, the implantable device is attached to one or more soft tissues within the breast region, described herein. Various methods of attachment are known in the art, and include, without limitation, suturing, stapling, gluing, and laying in place. Various attachment methods are described in U.S. Pat. No. 5,584,884.

The exact position of attachment will vary with the specific procedure being performed and can be determined by the skilled artisan. In one embodiment, attachment or fastening of the implantable device is to tissue surrounding the chest cavity of the patient. In one embodiment, attachment or fastening is to soft tissue surrounding the breast tissue and/or the prosthetic implant within the patient. The implantable device can alternatively be attached or fastened to a boney structure adjacent to the breast tissue and/or the prosthetic implant within the patient.

It may be beneficial or necessary for the skilled practitioner to form the implantable device into a predefined shape that is adapted to conform to a region, or at least a portion, of the natural breast tissue and/or the prosthetic implant within the patient. Such useful shapes include, without limitation, circular shapes, oval shapes, crescent shapes, cup shapes, and elongated strips.

It may be beneficial to treat the implantable device with one or more agents that promote cellular in-growth.

### Breast Augmentation

In one embodiment, the implantable device described herein is used as a surgical tool in breast augmentation. “Breast augmentation” as the term is used herein, refers to increasing the size of a breast, such as is generally achieved by the insertion of prosthetic implants.

The implantable device of the instant invention can be used to promote wound healing and soft tissue reconstruction by providing strength and covering at the site of a surgical incision (e.g., at the site of breast implant insertion). It can provide immediate strength to an incision site or site of soft tissue reconstruction/augmentation, and also provide a substrate for new tissue in-growth.

In one embodiment, the implantable device described herein is used in placement or repositioning of a breast prosthesis. The implantable device, for example, can be used to support the lower pole position of breast implants or can be used as a partial or complete covering of the breast implant. Covering of the implants within the implantable device provides a beneficial interface with host tissue and reduces the potential for malpositioning or capsular contracture. Covering of the implant also reduces or prevents tissue adhesions to the implant. Ultimately the implantable device can be absorbed and replaced by the infiltrating tissue. As such, the fabric can provide temporary scaffolding and well-defined structure until it is no longer needed.

The implantable device of the present invention can be used to reposition a breast implant in follow-up corrective surgery, or can be used prophylactically at the time of initial implant placement to prevent displacement. The implantable device can be configured and implanted to position the breast implant in the desired position within the patient (e.g., in completely sub-muscular, partial sub-muscular, or sub-glandular placement).

Implants are typically positioned within the chest in one of three positions: (1) implant over the pectoralis major muscle and under the breast tissue (subglandular); (2) implant partially under the muscle (partial submuscular); and (3) implant completely under the muscle (submuscular). The subglandular placement puts the implant directly behind the breast tissue and mammary gland and in front of the pectoralis major muscle. This placement requires the least complicated surgery and yields the quickest recovery. The downsides of this placement are increased chances for capsular contracture, greater visibility and vulnerability for the implant. This is because only the skin and breast tissue separate the implant from the outside world. Depending on the amount of available breast tissue, the implant may be seen “rippling” through the skin.

Partial submuscular placement involves placing the implant under the pectoralis major muscle. Because of the structure of this muscle, the implant is only partially covered. This alternative reduces the risk of capsular contracture and visible implant rippling, but recovery time from this positioning is typically longer and more painful because the surgeon has to manipulate the muscle during surgery. Also, because of increased swelling, the implant may take longer to drop into a natural position after surgery. Completely submuscular placement puts the implant firmly behind the chest muscle wall. The implant is placed behind the pectoralis major muscle and behind all of the supporting fascia (connective tissue) and non-pectoral muscle groups. This placement has even longer recovery time, potential loss of inferior pole fullness, and involves a more traumatic surgical procedure.

Regardless of location of the implant, in the case of breast augmentation the surgery is carried out through an incision placed to minimize long-term scarring. The incision is made in one of three areas: (1) peri-areolar incision; (2) inframammary fold incision; and (3) transaxillary incision. The peri-areolar incision enables the surgeon to place the implant in the subglandular, partial submuscular or completely submuscular position, with the implant being inserted, or removed, through the incision. Like the peri-areolar incision, the inframammary fold incision provides for all three placement types and both insertion and removal of the implant through the incision. The incision is made in the crease under the breast, allowing for discreet scarring. Once the incision is made, the implant is inserted and worked vertically into place.

Presently, there are very few techniques to reliably maintain the position of implants placed as part of cosmetic or reconstructive surgical procedures. Implant malposition may be the result of several factors, including poor surgical technique, i.e. the implant pocket is too big or too low; implant weight; or lack of soft tissue support. In addition, in reconstructive patients cancer treatments, such as chemotherapy, weaken the soft tissue and surgery, in general, interrupts the natural anatomic plains of the soft tissue. These factors are more profound in patients who have lost excessive amounts of weight. Such situations typically provide extremely poor soft tissue support and the inability of the usual support structures within the breast, such as the inframammary fold, to support the weight of the implant.

In one embodiment, the implantable device described herein is implanted within a patient for initial positioning of a breast implant within the patient. In such an embodiment, the implantable device may be configured to form a receiving area for receiving the breast implant. The implantable device may further comprise one or more regions for tissue affixation. One of the regions may be adapted to attach the implantable device to soft tissue surrounding the breast implant or a boney structure within the patient, such as the periosteum of the chest cavity, with a first suture or by conventional or endoscopic tacking.

During implant positioning or repositioning procedures the surgeon can use the initial incision made to insert the implantable device, provided the initial incision was peri-areolar or in the inframammary fold, to access and position the implant with the implantable device. However, in certain circumstances, such as if the initial incision is in the transaxillary position, it may be necessary to create a new incision. Once the incision is made, the implantable device can be inserted into the body through the incision.

The implantable device of the present invention can be configured to be implanted within the patient in varying orientations, depending on the specific situation to be remedied or prevented. For example, when used to correct medial displacement (symmastia) or lateral displacement of an implant, the implantable device is positioned in a substantially vertical position on the medial or lateral side, respectively, of the implant. When the implantable device is used to correct inferior displacement of an implant (otherwise known in the art as bottoming out), the implantable device is placed in a substantially horizontal position, supporting the implant from below. Proper positioning of the implantable device during the initial implant placement procedure is dependent on the tissue structure surrounding the implant and the desired placement of the implant within the patient.

Fixation of the implantable device is achieved, for example, by placement of permanent sutures at key locations via the tissue affixation regions, or with use of a tacking device, either conventional or endoscopic, depending on the placement of the incision. An inframammary fold incision may require suturing of the fabric in place whereas a peri-areolar incision will enable the use of an endoscopic tacking device.

When the implantable device is orientated in vertical position to fix or prevent medial displacement of the implant, the implantable device can be secured at tissue affixation regions to one or more of the following structures and soft tissue: 1) the backwall to the periosteum of the chest wall, 2) the upper intersection of the first and second portions to the sternal border of the chest wall, 3) at the lower intersection of the first and second portions to periosteum of the chest wall, and 4) on the frontwall to the posterior aspect of the pectoralis fascia.

### Mastopexy (Breast Lift)

Mastopexy, or breast lift, is a procedure designed to improve the appearance of sagging or ptotic breasts. Mastopexy presents one of the greatest challenges to the breast surgeon. Numerous techniques provide improvement in the shape of the breast, but aesthetic improvements comes at the cost of scars. In addition, the use of implants in mastopexy presents specific risks and complications. Four main types of breast lifts exist, crescent mastopexy, donut mastopexy, lollipop or vertical mastopexy and anchor mastopexy, based on the shape of the incision and the resulting scar.

Crescent mastopexy is for patients with mild sagging, excess breast skin in the upper half of the breast, and a normal amount of skin in the lower half, a semi-circular incision is made on the upper portion of the areola. A crescent shaped piece of skin is removed, and when the skin edges are sewn back together, the nipple and areola are raised slightly (1 to 2 inches). A crescent mastopexy is best for women with only mild breast ptosis (sagging).

Donut mastopexy, also called a Benelli mastopexy or circumareolar mastopexy since the incision is around the areola, a donut mastopexy removes a ring of skin from outside the areola. Sutures are then placed around the areola and the skin is tightened like a purse string to lift the breast. Puckering of the skin may occur, and usually resolves on its own within a few months. The donut mastopexy is also useful for women with a projecting nipple/areola complex (sometimes called torpedo or missile shaped breasts), and can also be used to reduce the size of the areola at the same time.

Lollipop or vertical mastopexy, as the name implies, is when an incision for a lollipop mastopexy is made around the areola and then down the center of the breast to the inframammary fold. This technique is used for mild to moderate breast ptosis. As with the circumareolar or donut lift, the size of the areola may be reduced at the same time.

Anchor mastopexy, also referred to as a Wise pattern (or sometimes Weiss pattern) mastopexy, full breast lift, or inverted-T incision, is considered the traditional technique for breast lifting. The incisions are made around the areola, down the center of the lower portion of the breast and then across the breast in the inframammary fold. Like the donut and lollipop incisions, the areola can be made smaller at the same time. The resulting scar is in the shape of an anchor. Although the Wise pattern or anchor mastopexy used to be the standard, it is now usually reserved only for those with moderate to severe breast sagging.

Mastopexy can be performed with or without a corresponding change in the breast size (either breast reduction or breast augmentation).

The implantable device described herein can be used in any of these types of procedures. In one embodiment, the implantable device described herein is used to promote wound healing and/or tissue support in the procedure. The implantable device can be also be used to augment or replace pre-existing breast tissue.

In one embodiment, the implantable device is used in a method to reduce breast volume. By way of non-limiting example, the method can be performed as follows:

1. Marking four points on the breast around the areola to determine the amount of skin necessary for both the external skin lining of the new breast and the excess skin in the periareolar region for the dermal flap to be used for the internal skin lining.

2. De-epithelializing the flap to retain the central pedicle.

3. Displace the breast subcutaneous down to the level of the pectoral fascia.

4. Dissecting the skin on the bias in the upper hemisphere in order to progressively increase the thickness of the subcutaneous fat tissue close to the skin.

5. Resecting a central wedge of tissue and shortening the upper hemisphere ray.

6. Dissecting the skin from the parenchymal tissue in the lower hemisphere of the breast.

7. Optionally resecting a second central wedge of tissue in the lower hemisphere.

8. Applying the appropriately shaped fabric over the dermal flap in the lower hemisphere to sling the underside of the breast.

9. Suturing the fabric to the pectoralis fascia to promote elevation and shape of the mammary cone.

10. Suturing closed the external skin lining while fixing the areolar skin to the external skin lining.

11. Dressing the breast in a supportive way that allows drainage of exudates.

In another embodiment, the implantable device is used in a method to lift breast tissue. By way of non-limiting example, the method can be performed as follows:

1. Marking four points on the breast around the areola to determine the amount of skin necessary for both the external skin lining of the new breast and the excess skin in the periareolar region for the dermal flap to be used for the internal skin lining.

2. De-epithelializing the flap to retain the central pedicle.

3. Displace the breast subcutaneous down to the level of the pectoral fascia.

4. Dissecting the skin on the bias in the upper hemisphere in order to progressively increase the thickness of the subcutaneous fat tissue close to the skin.

5. Dissecting the skin from the parenchymal tissue in the lower hemisphere of the breast.

6. Applying the appropriately shaped fabric over the dermal flap in the lower hemisphere to sling the underside of the breast.

7. Suturing the fabric to the pectoralis fascia to promote elevation and shape of the mammary cone.

8. Suturing closed the external skin lining while fixing the areolar skin to the external skin lining.

9. Dressing the breast in a supportive way that allows drainage of exudates.

In another embodiment, the implantable device is used in a method of mastopexy treatment with breast augmentation. By way of non-limiting example, the method can be performed as follows:

1. Inserting a breast implant either under the muscle in a submuscular pocket where the implant is large and the degree of sagging is greater, or under the breast gland in a subglandular pocket if the implant is small.

2. Marking four points on the breast around the areola to determine the amount of skin necessary for both the external skin lining of the new breast and the excess skin in the periareolar region for the dermal flap to be used for the internal skin lining.

3. De-epithelializing the flap to retain the central pedicle.

4. Displace the breast subcutaneous down to the level of the pectoral fascia.

5. Dissecting the skin on the bias in the upper hemisphere in order to progressively increase the thickness of the subcutaneous fat tissue close to the skin.

6. Dissecting the skin from the parenchymal tissue in the lower hemisphere of the breast.

7. Applying a mastopexy prosthesis over the dermal flap in the lower hemisphere to sling other underside of the breast.

8. Suturing the mastopexy prosthesis to the pectoralis fascia to promote elevation and shape of the mammary cone.

9. Suturing closed the external skin lining while fixing the areolar skin to the external skin lining.

10. Dressing the breast in a supportive way that allows drainage of exudates.

### Breast Reconstruction

Breast reconstruction is the re-creation of a breast following mastectomy. Mastectomy is the most common treatment of localized breast cancer. While breast reconstruction can be performed at the time of mastectomy, the better candidates are those who have confirmed elimination of the cancer as sometimes implant materials and reconstruction will interfere with detection of recurrence. Reconstruction usually involves a two part process, where in the first series of surgeries, a tissue expander is inserted beneath the skin and the pectoralis muscle. The expander is an air or saline-filled balloon that is periodically injected over a number of months with additional saline in order to gradually stretch the skin and muscle. When the skin and muscle are sufficiently lengthened, an implant (saline or silicone) is inserted to recapitulate the native breast structure. However, in order to retain the implant properly, an additional section of a patient's tissue, an autograft, must be used along the lateral side of the breast, usually the latissimus dorsi or abdominus recti. Autograft tissue bears a risk of tissue morbidity and total coverage and support of the implant or the expander with the muscle tissue in the mastectomy pocket is a challenge. Without appropriate coverage, the implant can become exposed and reduce cosmetic outcome.

The implantable device described herein can be used to promote wound healing and/or tissue support in the procedure. The implantable device can also be also be used to augment or replace pre-existing breast tissue. The implantable device can further be used in implant placement as described herein in the breast reconstruction procedure. In one embodiment, the fabric described herein is used in complement or in place of autograft tissue in the breast reconstruction procedure (e.g., to cover and/or support the implant or the expander at the lower breast pole).

In one embodiment, the implantable device of the present invention is used to provide strength to breast fascia and/or soft tissue weakened by the mastectomy surgery. During mastectomy, as much of the superficial fascial system in the inframammary fold is preserved as possible. Generally, Cooper's ligaments are cut in the course of the surgery. In one embodiment the implantable device of the present invention is used to recreate the inframammary fold following mastectomy. In one embodiment, the implantable device of the present invention is designed to have one or more biomechanical properties of the inframammary fold tissue that is damaged during the mastectomy process. This implantable device can be implanted at the location of the damaged tissue. Such implanted device supports the reconstructed breast and also serves as a scaffold for the generation of new tissue at that site within the body.

In one embodiment, the implantable device of the present invention can be used in place of, or in combination with, the omental flap, in postmastectomy breast reconstruction. One such procedure is described by Goes and Macedo (The Surgery of the Breast, Principles and Art, Lippincott Williams & Wilkins, Second Edition, Chapter 52, pages 786-793, 2006). In another embodiment, the implantable device can be used for nipple reconstruction. The implantable device may incorporate a shaped structure to provide support for a nipple reconstruction.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It will be apparent to those skilled in the art that many modifications, both to the materials and methods, may be practiced without departing from the invention.

While the present invention has been described in connection with a number of exemplary embodiments, and implementations, the present inventions are not so limited, but rather cover various modifications, and equivalent arrangements, which fall within the purview of prospective claims. All dimensions, measurements, and the like, provided herein are exemplary.

What is claimed is:

1. A method of supporting breast tissue or a breast implant in a patient comprising, providing an implantable device for a breast augmentation or a breast reconstruction procedure, the implantable device comprising:
at least one knitted section, and
at least one single continuous fiber traversing the at least one knitted section, the at least one single continuous fiber forming a plurality of traverses extending through the at least one knitted section, and
inserting the implantable device between skin of the patient and the breast tissue or the breast implant.

2. The method according to claim 1, further comprising fastening the implantable device to tissue surrounding the chest cavity of the patient.

3. The method according to claim 1, further comprising fastening the implantable device to soft tissue surrounding the breast tissue or the prosthetic breast implant.

4. The method according to claim 1, further comprising fastening the implantable device to a boney structure adjacent to the breast tissue or the prosthetic breast implant.

5. The method according to claim 1, further comprising forming the implantable device into a predefined shape adapted to conform to at least a portion of a region of natural breast tissue or a breast implant.

6. The method according to claim 1, further comprising treating the implantable device or support structure with a factor for promoting in-growth of breast tissue.

7. The method according to claim 1, further comprising inserting in an inframammary region of the breast, a medial side of the breast, or in a lateral side of the breast.

8. The method according to claim 1, wherein the at least one single continuous fiber is silk.

9. The method according to claim 8, wherein the silk is Bombyx mori silkworm silk fibroin.

10. The method according to claim 9, wherein the silk fibroin is a sericin-extracted silk fibroin fiber that substantially retains its native protein structure and has not been dissolved and reconstituted.
